(19) United States
(12) Patent Application Publication
(10) Pub. No.: US 2005/0208187 A1
(43) Pub. Date: Sep. 22, 2005

(54) Process and apparatus to inject fluids

(76) Inventor: William R. Kowalski, Honolulu, HI (US)

Correspondence Address: Martin E. Hsia, P.O. Box 939, Honolulu, HI, 96808, US

(21) Appl. No.: 10/804,789

(22) Filed: Mar. 19, 2004

(51) Int. Cl. A61M 37/00

(52) U.S. Cl. 426/281

(57) ABSTRACT

A process and apparatus for injecting fluids through needles at controlled mass flow rates into permeable solids such as meat or fish. This invention utilizes a multiplicity of needles, each having a flow control orifice that is sized to non-mechanically control the mass flow of fluid through the orifice. When fluid at a fixed pressure is applied to the inlet of each orifice, fluid mass flow through each needle remains constant with increases in backpressure until the backpressure reaches approximately one-half of the inlet pressure. The rate of fluid mass flow through a needle is calibrated to discharge a particular fluid volume at a particular inlet pressure and backpressure. When the needles are inserted into a permeable solid having regions of varying densities, resistances or permeability, the change in rate of fluid mass flow is minimized over such regions to cause even distribution of fluid throughout the permeable solid.

### TECHNICAL FIELD

This invention relates to a process and apparatus to inject fluids into solids. A variety of gases, liquids, and combination fluid substances can be applied to influence the taste, appearance, and microbial condition of solid food products. Fluids can be injected and absorbed into porous meats causing various treatment effects. For example, salt solutions can be applied to meats imparting taste, improving texture, and extending microbial shelf life. Tripolyphosphate can be added to seafood for weight gain. Carbon dioxide gas can be dissolved into flesh and increase acidity thereby reducing microbial activity. Carbon monoxide gas can be applied to influence color and prevent oxidation of meat. Ozone in gaseous vapor or liquid form can be applied to reduce bacteria. Binding fluids can be injected to form meat from trimmings. Other gaseous and liquid fluids can be blended to form vapors or colloid solutions for injection for other treatment effects.

Fluid penetration into meat by external exposure can be slow when applied to thick meat cuts and can result in loss of freshness as well as uneven or incomplete treatment. The present invention utilizes hypodermic needles to inject fluids into meats, thereby reducing treatment time and providing even distribution of fluids throughout the interior of the meat. Reduced treatment time promotes meat freshness and food processing efficiencies. The present invention provides a superior process to precisely control the mass flow of fluids through needles during injection of meats, especially the mass flow of gases through needles that are very difficult to accurately control in small consistent dosages suitable for meats, especially seafood.

### BACKGROUND ART

Hollow needles similar to hypodermic needles can be used to inject fluids, gaseous or liquid, preferably into permeable solids such as meat (including fish) for the purpose of treating the entire volume of the permeable solid.

In the following explanations, gas flow into meat (including fish) has been used as an example. Certain characteristics of the present invention are unique to gas, however the process accurately controls the constant flow of any fluid into any permeable solid. This feature is of particular value when fluid types of gas and liquid are combined.

For example, an apparatus for injecting gas into fish would include a multiplicity of hollow needles connected to a source of gas under pressure, a valve for controlling the gas flow to the needles, and a means for causing the needles to penetrate the fish. The penetration and gas flow can be continuous until the entire volume of the fish has been treated, or intermittent, where the penetration and/or the gas flow are intermittent, continuing until the entire volume of the fish has been treated.

Significant problems have been encountered in previously designed apparatus for injecting gas into fish.

One problem is that fish have variable densities, permeabilities or resistances to gas flow i.e., they contain soft and hard regions. When a constant gas injection pressure is applied, the gas flow rate into the soft regions of the fish is high, causing damage to the fish meat, and the gas flow rate into the hard regions of the fish is low, resulting in insufficient treatment of the fish. These regions of varying density are randomly distributed throughout the fish. Thus, with a multiplicity of needles, some needles may be in soft regions while others are in hard regions, making it impossible to overcome this problem by simply varying the constant gas injection pressure to all needles simultaneously.

Another problem is that a needle (or needles) may become plugged as it penetrates the fish by fragments of fish or liquids that coagulate in the needle, preventing gas flow from that needle into the fish. This can occur during continuous injection or during intermittent operation, especially when the gas flow is shut off during periods of penetration.

If this problem is overcome by increasing gas pressure, the previously described problem of damage to fish meat due to high gas flow rates is exacerbated. Even though the plugs can be removed by mechanical or other means after the needles have been withdrawn from the fish, the problem of plugging still results in untreated regions in the fish.

Another problem of previously designed apparatus is mechanical valve malfunction. If an independent mechanical valve is applied to each individual needle, then the likelihood of mechanical error is increased as needles and corresponding valves are multiplied.

Another problem is that the injection apparatus wastes gas unless the gas flow is started just as the needles start to penetrate the fish. With fish pieces of varying thickness and a fixed needle travel, there can be a considerable amount of wasted gas. This problem can be overcome by manually operating the gas valve at the instant the needles contact the fish, but this requires a skilled operator.

Various methods have been used to control a constant flow of liquids. However, the behavior of compressible gases is different than liquids. Therefore, the known methods for injecting liquids do not apply to injecting gas.

Yamaoka's “High efficiency preserving treatment method for fish meats being eaten raw”, HEI [1994] 334536 Dec. 19, 1994 teaches a process for injecting gas into fish. However, Yamaoka is limited to intermittent “bubbles” of gas injected at fixed intervals. Yamaoka's method cannot accurately control a constant flow of gas into fish as taught in the present invention.

Albright et al U.S. Pat. No. 2,645,172 Apr. 14, 1953 discloses an invention for injecting curing liquids into meats. Albright illustrates liquid passing through ducts to reduce pressure in the hollow needle to sustain a uniform amount of brine during injection. However, Albright does not apply to gas.

Marks Standard Handbook for Mechanical Engineers 8^{th} Edition Pages 4-46 and 4-47 “Flow Through Orifices and Nozzles” teaches general formulas for orifice computations. However, these principles have not been applied to needle injection.

Muller et al U.S. Pat. No. 4,903,590 Feb. 27, 1990 discloses a process to control liquid volume during injection by dividing the needles into groups. Pressure is controlled and adjusted for each group of needles depending on the meat characteristics. According to the present invention it is not necessary to adjust pressure in various needle groups to maintain constant fluid flow volume in each individual needle.

Townsend U.S. Pat. No. 4,292,889 Oct. 6, 1981 discusses a method for injecting meat by metering fluid (i.e. liquid) to a precise volume equal to the cavity formed by the needle. However, Townsend's metering device is directed to a measured amount of fluid delivered to a group of needles, while the present invention is directed toward controlling flow through each individual needle.

Lumby et al U.S. Pat. No. 3,814,007 Jun. 4, 1974 teaches an apparatus that allows fluid pressure to build to a predetermined level before injection commences. Lumpy is limited to intermittent injection of liquid. However, the present invention permits continuous fluid injection, which may be desired.

Smith U.S. Pat. No. 5,773,060 Jun. 30, 1998 teaches a method of injecting fluid into meat or fish, by adjusting the fluid (i.e. liquid) volume injected into the meat to a target ratio of meat to fluid. However, Smith only controls fluid pressure to a group of needles. Smith does not provide a means to control the discharge flow of gas through individual needles.

Wallace U.S. Pat. No. 4,551,338, Nov. 5, 1985 teaches a method for changing the internal temperature of meat by the use of gas. However, Wallace is not concerned with controlling a specific rate of gas flow through each needle.

Townsend U.S. Pat. No. 3,863,556 Feb. 4, 1975 teaches a means to sense the thickness of meat during injection and adjusting the fluid volume accordingly. However, Townsend does not teach any method to control precise gas flow through individual needles.

Townsend U.S. Pat. No. 4,142,000 Feb. 27, 1979 reveals the use of a stripper plate in combination with a method to adjust the flow of fluid through the needles to a rate that is proportionate to the penetration rate of the needles during injection. However, no means to manage precise discharge volume of fluid from individual needles is disclosed.

Townsend U.S. Pat. No. 5,996,481 Dec. 7, 1999 teaches a method to inject fluid into meat as the meat is moving, plus to permit the fluid to flow continuously and return to the reservoir when the needles are not in the meat. However, the invention does not teach a means to control a stable gas flow through individual needles.

Townsend U.S. Pat. No. 4,254,151 Mar. 3, 1981 makes known a method to inject fluid that would be uniformly distributed through the meat. However, uniformity is based on the total quantity of fluid injected through a group of needles and the method fails to control a measured mass flow of gas through each individual needle.

Townsend U.S. Pat. No. 4,455,928 discloses a plurality of individual fluid manifolds with needle groups. A source regulates fluid flow independently to each individual manifold, which operates in combination with a stripper plate as a fluid valve. However, the method does not restrict the difference in fluid flow between the individual needles caused by varying resistance to flow within the meat.

### SUMMARY OF THE INVENTION

The present invention is a new method for injecting fluids through needles. Each individual needle has an orifice that is sized to control the mass flow of the fluid used—either a gas, a liquid or a combination. When gas at a fixed pressure is applied to the inlet of the orifice, the gas mass flow through the needle remains constant with increases in backpressure until the backpressure reaches approximately one-half the inlet pressure.

This invention primarily applies to a process and apparatus for injecting gas. However, this method is effective for controlling the mass flow of all fluids—either gasses, liquids, or their combinations—at controlled flow rates that are fit for meat, fish, or other permeable solids.

The rate of fluid mass (matter) flow through the needle is calibrated to discharge a particular fluid volume at a particular inlet pressure and backpressure. At a fixed inlet pressure gas mass flow remains constant, yet the gas volume varies inversely to backpressure.

Sufficient volumetric quantity of fluid is important for complete exposure of a permeable solid. However, excess volumetric quantity may cause damage. Therefore, it is appropriate to express fluid volume discharging the orifice/needle in cc at ambient pressure.

Multiplicities of the individual needles with flow control orifices can be connected to a manifold that provides a source of pressurized gas to all the needles. During injection, the mass flow of gas remains the same through each individual needle in the multiplicity, even if the density and back pressure of the permeable solid at each individual needle varies up to a back pressure of about one half the inlet pressure.

We have empirically discovered that the backpressure of the permeable solid at any individual needle must be greater than 50% of the source pressure in the manifold before there is any significant change in gas mass flow. For example, if the source pressure in the manifold is 200 P.S.I. (14.045 kg/cm²), then the backpressure of the permeable solid must be more than approximately 100 P.S.I. (7.022 kg/cm²) before any significant change in gas mass flow occurs.

We have observed references that support our discoveries. Marks Standard Handbook for Mechanical Engineers 8^{th} Edition, Pages 4-46 and 4-47, section entitled “Flow Through Orifices and Nozzles” states:
“The smallest cross section of the nozzle is called the throat, and the pressure at the throat is the CRITICAL FLOW PRESSURE . . . . If the nozzle is cut off at the throat with no diverging section, and the pressure at the discharge end is progressively decreased, with fixed inlet pressure, the amount of fluid passing increases until the discharge pressure equals the critical, but further decrease in discharge pressure does not result in increased flow . . . . For gasses, pm/pl=0.53.”

We have determined by experimentation and research that when gas pressure at the orifice entry exceeds approximately two times the pressure below the orifice, then the mass flow of gas through the orifice becomes independent of backpressure below the orifice. Therefore, as long as gas pressure above the orifice is maintained at more than about two times the pressure below the orifice (in absolute pressure), then gas mass flow through the orifice will remain at sonic velocity and flow will remain independent of back pressure below the orifice.

For liquids, back pressure below the orifice reduces flow through the orifice in all cases. However, it was surprising to discover that mass flow of a gas remains constant in the presence of back pressure below the orifice if the sonic velocity of the gas through the orifice is maintained. As a result, a constant mass flow of gas can be maintained with greater precision than a mass flow of liquid when injecting meat with variable resistances. This is an amazing discovery, because gas is more fluid than liquid and it is logical to expect gas flow to be more difficult to control than liquid flow during injection into permeable solids.

The characteristics of gas flow through orifices differ from the characteristics of liquid flow through orifices. Liquid flow through orifices varies with density, pressure, and shape of the orifice entrance. For very small orifices viscosity has a minor effect. The velocity of liquid flow through an orifice (neglecting viscosity) can be calculated by the formula V=square root of 2gh where g is the acceleration of gravity and h is the height of the column of liquid.

Gas flow through orifices follows the same formula up to the point where the flow reaches nearly sonic velocity. This occurs at about two times absolute ambient pressure, or approximately 14.7 P.S.I. (1.053 kg/cm²) of gauge pressure. Above this pressure, as long as the pressure below the orifice is less than half the pressure above the orifice, the velocity of gas flow through the orifice does not increase. The gas flow velocity remains at sonic velocity at about 1,100 feet per second (335.28 m/sec.).

Thus for a specific gas flow rate, each orifice must be sized for a specific gas (density) and for a specific pressure above the orifice (and below the orifice if the pressure below the orifices exceeds half of the pressure above the orifice.) This invention provides a simple and direct method of manufacturing orifices for desired flow rates.

For example, the flow of a liquid (e.g. water) through an orifice of 0.005 inch (0.127 mm) diameter and 0.0000196 sq. inch (0.0127 mm²) area at 50 P.S.I. (3.523 kg/cm²) is 0.0203 cu. in. per second (0.3326 cm³/sec.) and will vary with the square root of the ratio of new pressure to the current pressure.

The flow of a gas (e.g. air) through an orifice of 0.005 inch (0.127 mm) diameter at 50 P.S.I. (3.523 kg/cm²) will be at a sonic velocity of approximately 1,100 feet per second (335.28 m/sec.) and a pressure of approximately 33 P.S.I. (2.325 kg/cm²) (half of 64.7 P.S.I.) absolute in the orifice throat, giving a flow of 0.58 cu. in./second (9.5045 cm³/sec.) after expansion to ambient pressure.

Using as an example an apparatus for injecting gas into whole or cut pieces of fish, such an apparatus would include a multiplicity of hollow needles connected to a source of gas under pressure, a valve for controlling gas flow to the needles, and a means for causing the needles to penetrate the fish.

This invention provides in such an apparatus, a means for minimizing the change in rate of flow of gas mass into the fish when the fish varies in permeability or resistance to flow, i.e. from soft to hard regions, in the same fish.

This invention also provides in such an apparatus, a means for preventing blockage of needles by fragments of fish, or for removing existing blockages, without causing damage to the fish.

This invention also provides a novel means to manufacture an orifice to accurate flow limits by sizing the orifice while gas is flowing through the orifice, until the desired flow rate is reached. For very small orifices, this eliminates the necessity for mechanically creating an extremely small orifice, of the proper entrance contour, to very close dimensional tolerances.

Manufacturing orifices of a predetermined size of less than 0.01 inch (0.254 mm) in solid materials such as metals with tolerances less than +/− 0.001 inch (0.0254 mm) is highly difficult with irregular orifice shape and edges can be expected. Ideal gas flows for injecting gas into tuna requires orifices in the range of 0.0024 inch (0.06096 mm) to 0.0031 inch (0.07874 mm) diameter. If a tolerance of +/−0.001 inch (0.0254 mm) is applied to an orifice of 0.0027 inch diameter, the flow through the larger orifice is 3.6 times the flow through the smaller orifice, an unacceptably wide flow range. Using the orifice sizing method described in this invention, the flow variation is a function of the flow meter tolerance (about +/− 6%) and operator skill. In actual practice over several thousand orifices, we have found that the total flow variation can be kept to +/− 12%. This provides a flow variation between maximum and minimum of 1.27 times, which is acceptable.

It is therefore an object of this invention, in an apparatus for injecting fluids such as gas into a permeable solid such as fish through a multiplicity of hollow needles, to provide a means for minimizing the change in rate of flow of fluid when the permeable solid varies in density or resistance to flow throughout regions of the solid.

It is a still further object of this invention, in a process and apparatus for injecting gas into a permeable solid such as fish using a hollow needle, to provide a means to maintain sonic velocity of the gas through an orifice located at any point along the needle.

It is a still further object of this invention, in a process and apparatus for injecting fluids such as gas into a permeable solid such as fish using a hollow needle, to provide a means to maintain sonic velocity of the gas through an orifice where such orifice is the interior diameter, or I.D. of the needle.

It is a still further object of this invention, in a process and apparatus for injecting fluids such as gas into a permeable solid such as fish through a multiplicity of hollow needles, to have the pressure above the orifice be at least 50% greater than the pressure below the orifice.

It is a still further object of this invention, in a process and apparatus for injecting fluids such as gas into a permeable solid such as fish through a multiplicity of hollow needles, to provide a means for preventing or removing blockages of needles used for injection without causing damage to the permeable solid being treated by fluid injection.

It is a still further object of this invention, in a process and apparatus for injecting fluids such as gas into a permeable solid such as fish through a multiplicity of hollow needles, to provide a means for minimizing the change in rate of mass flow of fluid when the permeable solid varies in permeability, density, or resistance to flow by providing a high pressure source of fluid such as gas, in combination with a flow controlling orifice applied to each needle.

It is a still further object of this invention, in an apparatus for injecting fluids such as gas into a permeable solid such as fish through a multiplicity of hollow needles, to provide a means to eliminate mechanical failure, by incorporating a non-mechanical orifice to control flow into each individual needle.

It is a still further object of this invention, in an apparatus for injecting fluids such as gas into a permeable solid such as fish through a multiplicity of hollow needles, to provide a means for automatically starting the gas flow at the time the needles start to penetrate the fish by providing a moveable device, such as a moveable needle guide, which causes gas flow to begin when the device contacts the fish.

It is a still further object of this invention to incorporate the orifice in the needle, at any location along the needle or running the entire length of the needle, or independent of the needle yet still able to control flow through the needle

It is a still further object of this invention to incorporate multiple orifices within a needle or in conjunction with a needle.

Other objects of this invention are referenced in the description of the best mode of practicing the present invention and its drawings:

### BRIEF DESCRIPTION OF THE DRAWINGS AND CHARTS

FIG. 1 is a graphical illustration of an apparatus for injecting gas into a permeable solid, such as fish, through a multiplicity of hollow needles.

FIG. 2 is a cross sectional view through a portion of a group of needles showing the gas chamber, needles, and flow control orifices.

FIG. 3 is a graphical illustration of the pressure relationships above and below the flow control orifice.

FIG. 4 is a cross sectional view through the end of a single needle, with the needle plugged by a fragment of fish, which prevents flow of gas through the needle.

FIG. 5 is a graphical illustration of the pressure, flow, and time relationships before and after the plug is ejected by increasing gas pressure.

FIG. 6 is a graphical illustration of a multiplicity of needles in a gas manifold with a gas delivery system and hydraulic drive system for injection into fish.

FIG. 7 is a cross sectional view of a fluid injection assembly, showing the gas chamber, needles, and needle guide plate.

FIG. 8 is an illustration of a method to manufacture a flow control orifice in a needle.

### BEST MODE OF PRACTICING THE INVENTION

The best mode of practicing the invention is to group individual needles into a multiple needle injection machine used for injecting gas into fish. Preferably, an orifice is incorporated in the body of each individual needle. One reason this is preferred is that it minimizes the size of the gas cavity between the orifice and the discharge tip of the needle, thereby increasing the speed of pressure increase and decrease in each needle when injecting permeable solids of variable back pressure. Increases in back pressure of the permeable solid will cause pressure rise after the orifice in the needle until source pressure is reached. Decreases in the back pressure of the permeable solid will decrease the pressure in the needle cavity below the orifice.

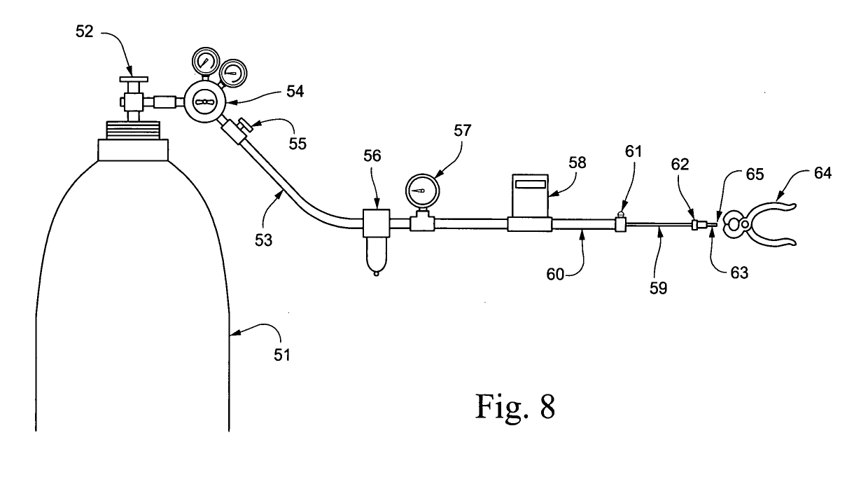

The orifice manufacturing procedure shown in FIG. 8 consists of a gas or fluid source (51) with a valve (52) connected by a pipe (53) to a two-stage pressure regulator (54), shutoff valve (55), and filter (56). A pressure gauge (57) monitors the pressure of the fluid leading into the flow meter (58) and then to the hollow tubing (59) that forms the needle. Any means to measure the mass flow can be used. For the present example a Cole Parmer brand Gas Mass Flowmeter was selected. The discharge end (during injection) of the needle (59) is inserted into a flexible hose (60) and clamped (61). All connections are airtight.

Preferably, the needle hub (62) is connected to the needle (59) before beginning the orifice manufacturing procedure. A section of needle (63) extends beyond the needle hub (62) and this needle section (63) will be crimped by a crimping tool (64) as later explained. The end of the needle (63) is the inlet end of the needle (59) during injection.

For purposes of this illustration the needle is 8 inches (203.2 mm) long, 0.040 inch (1.016 mm) outside diameter, and a 0.020 inch (0.508 mm) inside diameter and the gas flow through the needle (59) is 1 cc per second at 20 P.S.I. (1.404 kg/cm²) (57). However, the fluid flow can be controlled to any desired rate, even a fraction of 1 cc per second at much higher fluid pressure. A desired fluid source (51) is selected. Gas is used in the forgoing examples; however any fluid or combination of fluids can be used. The valve (52) and shutoff valve (55) are opened to supply gas from the source (51) and the pressure regulator (54) is adjusted until the pressure gauge (57) indicates 20 P.S.I. (1.404 kg/cm²). An orifice sizing means is used to calibrate the needle to a specific fluid flow at a specific fluid pressure. Using the crimping tool (64), pressure is applied to the needle end (65) to collapse the hollow tubing and reduce the orifice cross sectional area perpendicular to the needle shaft until the flow meter (58) measures 1 cc per second. The orifice at the end of the needle (65) is now complete, the shutoff valve (55) is closed, and the needle (59) is removed. This is one example of orifice production that achieves an accurate flow rate. However, any means to accurately size the orifice can be used. Another example is to collapse the needle end (65) to a pencil point and then to remove material with a grinder to expose the hollow area of the needle and increase the orifice size until the flow meter (58) reads the desired fluid flow rate.

Other methods can be used for orifice production such as hole punching, drilling, and other mechanical means of producing holes. However, non-mechanical means such as small hole EDM (Electrical Discharge Machining), wire EDM, and LASER (Light Amplification by Stimulated Emission of Radiation) can be used for ultra precision hole production. All of these methods have tolerances in hole size, hole shape, surface texture, and hole entrance shape that can substantially affect the fluid flow, especially gas flow at high pressure.

According to the present invention, orifice tolerances are irrelevant to flow, when the orifice is sized with fluid flowing through the orifice. Additionally, the cost is greater to produce orifices to a predetermined specification with either mechanical or non-mechanical means, yet the resulting flow accuracy is less then the present method. However, flow accuracy can be increased and cost reduced by applying mechanical or non-mechanical means in conjunction with the method described herein of sizing the orifice while fluid is flowing through the orifice. Control of fluid flow through needles using the method described herein is not restricted to any particular type of liquid, orifice size, pressure range, or number of needles, since these parameters can be adjusted according to the desired application. Multiple orifices can be combined in individual needles and multiple needles can be fed from individual or multiple orifices.

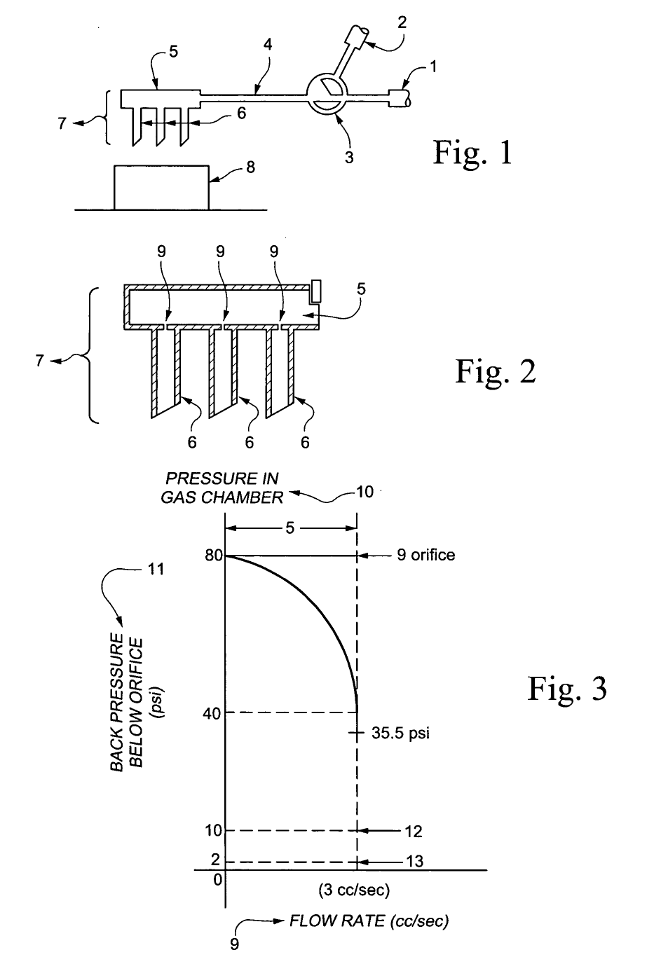

Referring to FIG. 1, a 3-way valve (3) directs pressurized gas from a source (1) into the pipe (4) leading into a gas chamber (5) with needles (6) as outlets. The needles are pressed into the permeable solid (8), which for purposes of this illustration is fish. However any permeable solid applies. A suitable amount of gas (1) is injected into the fish (8). After gas (1) injection is complete, the 3-way valve (3) is activated to shut off the gas (1) and release the pressurized gas in the chamber (5) through the pipe (4) and out an exhaust port (2).

FIG. 2 illustrates a cross sectional view of a portion of the needle assembly (7), showing the flow control orifice (9), between the gas chamber (5), and the needle (6). The orifice is shown at the top of the needle. However it can be at any location along the length of the needle. Alternatively, the internal area of the needle can be sized so that the entire needle length acts as an orifice, or an orifice means can be located before the needle. The size and shape of the orifice allows the desired gas mass flow rate when high-pressure from the source (1), is applied above the orifice (9).

FIG. 3 is a graph showing the back pressure below the flow control orifice as the ordinate (11) given a constant gas pressure (10) in the gas chamber (5) above the orifice, and mass gas flow in cc/sec as the abscissa. When the constant gas pressure (10) in the gas chamber (5) is 80 P.S.I. (5.618 kg/cm²) gauge and 94.7 P.S.I. (6.65 kg/cm²) absolute, and the back pressure is equal to this constant gas pressure (10) above the orifice, then there is zero mass gas flow.

As the back pressure decreases from 80 P.S.I. (5.618 kg/cm²) gauge and 94.7 P.S.I. (6.65 kg/cm²) absolute, the flow through the orifice increases until the back pressure below the orifice reaches the “critical pressure.” This critical pressure is computed by Marks in the Mechanical Engineers Handbook to be approximately 0.53 times the absolute pressure above the orifice, when the flow through the orifice reaches sonic speed. When the back pressure is below the critical pressure, the flow does not change, regardless of back pressure. In this example, the flow rate after the back pressure declines to the critical pressure or below is a constant 3.0 cc/second.

Using Mark's factor of approximately 0.53 times the absolute pressure of 94.7 P.S.I. (6.65 kg/cm²) above the orifice establishes the critical pressure level for back pressure of 50.2 P.S.I. (3.524 kg/cm²) absolute or 35.5 P.S.I. (2.492 kg/cm²) gauge pressure or below to achieve the constant mass gas flow of 3.0 cc/second.

FIG. 3 also shows for illustration purposes, the same constant mass gas flow of 3.0 cc/second for back pressures of 10 P.S.I. (0.702 kg/cm²) in the hard fish regions (12), and 2 P.S.I. (0.14 kg/cm²) in the soft fish regions (13). These pressures have been chosen as typical only to illustrate the flow relationships, as actual pressures vary widely between varieties of fish. Thus there is no change in gas flow, expressed as cc per second at atmospheric pressure, when the back pressure is below 35.5 P.S.I. (2.492 kg/cm²).

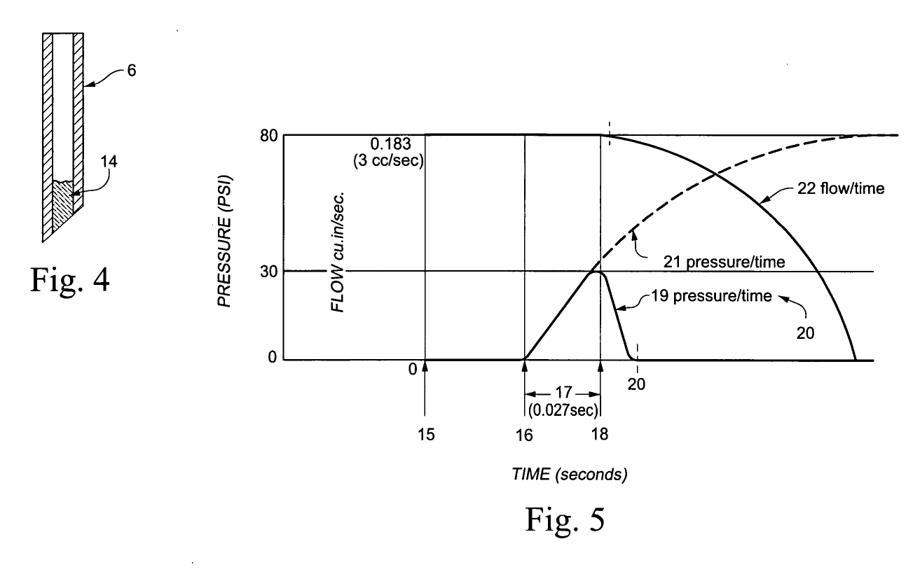

FIG. 4 illustrates that when a needle strikes a small region of very high back pressure, such as a fiber, the needle may become plugged by a fragment of fish, as is shown in the sectional view of the end of a needle (6), with a fragment of fish (14) plugging the needle and preventing gas flow.

FIG. 5 is a graph having gas pressure and gas flow rates as ordinates, and time as the abscissa. It shows the time before plugging (15), the time at plugging (16), the period of time for pressure build-up while plugged (17), the time of plug ejection (18), and changes in pressure (19), and flow (20), after plug is ejected. Also shown are the changes in pressure (21), and flow (22), for a permanent, unremoveable plug.

FIG. 5 shows the time-pressure relationships during such a plugging period. Assuming a back pressure of 30 P.S.I. (2.110 kg/cm²), the time for the gas pressure to build to 30 P.S.I. (2.110 kg/cm²) gauge, in a needle having an inside diameter of 0.020 inches (0.508 mm) and a length of 8 inches (203.2 mm), with an internal volume of 0.0025 cubic inches (40.87 mm³), at an initial flow rate of 3 cc per second, equal to 0.183 cubic inches (2998.83 mm³) per second of gas at atmospheric pressure, will be 0.0025 per 0.183×30/14.7=0.027 seconds (40.87 per 2998.8×2.1098/1.0338=0.027 sec.). With a needle penetration rate of 2 inches per second (50.8 mm/sec.), the length of the plug will be 2×0.027=0.054 inches (50.8×0.027=1.3716 mm). As the operation continues, the pressure will rise toward 80 P.S.I. (5.626 kg/cm²) and the plug may be ejected.

The amount of gas discharged into the fish at plug ejection will be 0.0025×30/14.7=0.005 cubic inches (0.0409×2.1098/1.0338=0.0835 cm³). Even at 80 P.S.I. (5.626 kg/cm²) the amount of gas discharged into the fish at plug ejection would be only 0.0025×80/14.7=0.0136 cubic inches (0.0409×5.6261/1.0338=0.223 cm³). This small volume of gas is insufficient to damage the fish.

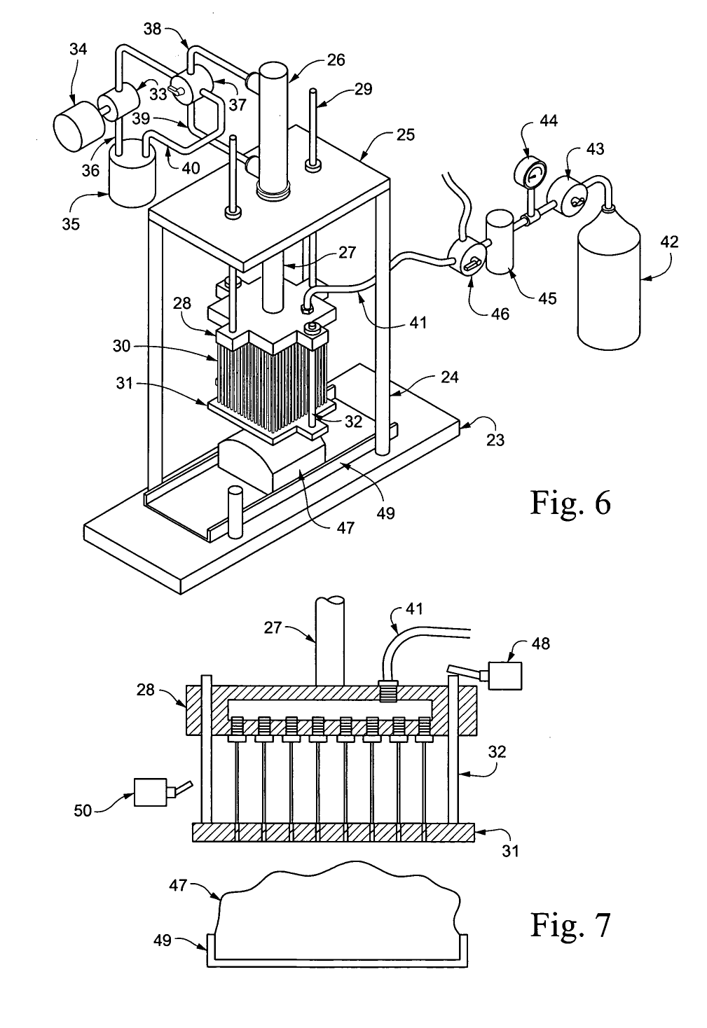

Injection of fluids into permeable solids can be done using a machine as illustrated in FIG. 6, although the fluid pressure, orifice size, flow rate, and other parameters would be chosen to suit the fluid being injected and the permeable solid being treated.

The machine shown in FIG. 6 has the following principal elements:

A base (23), an injection support frame (24), a hydraulic cylinder support (25), and a hydraulic operating cylinder (26).

The piston rod (27), extending downward from the hydraulic cylinder (26), supports and drives a gas chamber (28), gas chamber guide rods (29), a needle group (30), a needle guide plate (31), and needle guide plate guide rods (32),

The needle group (30), of FIG. 6 consists of 8 rows of needles with 8 needles in each row. The needles are 1 cm (“centimeter”) apart, so this needle group will treat a section of fish 8 cms square. Each needle is 20.3 cubic meters (8 inches) long, 1 mm outside diameter, ½ mm (0.020 inches) inside diameter, with an orifice at its upper end equivalent to 0.057 mm diameter.

At an input gas pressure above the orifice of 80 P.S.I. (5.618 kg/cm²) gauge, the gas flow into the fish will be approximately 3 cc per second of gas at atmospheric pressure per needle.

If gas pressure is increased, then the orifice size must be decreased, in order to maintain the same gas flow rate. Reducing the orifice size increases the variability of the machine operating parameters such that a higher gas pressure and slower needle penetration speeds can be used.

Orifice size, fluid pressure, and fluid discharge volume from the needle are in relationship with the rate that needles penetrate the permeable solid. When needle penetration rate is reduced, then fluid discharge volume can be reduced to maintain the same total volume of fluid injected. When needle penetration rate is increased, then fluid discharge volume can be increased to maintain the same total volume of fluid injected. Fluid volume can be decreased by decreasing the orifice size and/or the fluid pressure. Conversely, fluid volume can be increased by increasing orifice size and/or the fluid pressure.

In a functioning injection system, it is preferred to decrease fluid flow by reducing the orifice size and increase fluid flow by increasing the fluid pressure, which maintains or improves the fluid flow stability through the needle. In this manner, multiple fluids (for example a gas and a liquid) can be applied independently or in combination through the same needle system, while the fluid flow rate is controlled simply by adjusting the fluid pressure. We have not found any specific limitations as to the minimum orifice size, maximum fluid pressure, or minimum fluid flow rate. Indeed operable orifices can be smaller than 0.0005 inch (0.013 mm) diameter (which may be outside the capability of any other technology), fluid pressures can exceed 2,000 P.S.I. (14.45 kg/cm²), and fluid flow can be less than 0.05 cc per second. Variability to regulate a wide range of fluids including gases, liquids or combinations such as colloids and vapors, to accurate flow limits is a highlight of the present process and apparatus.

The needle penetration rate during injection of fluids measured in inches of needle travel per second optimally range between approximately 1 inch per second (25.4 mm/mm/sec.) and approximately 10 inches per second (254 mm/sec.), preferably ranges between approximately 0.5 inch per second (12.7 mm/sec.) and approximately 24 inches per second (609.6 mm/sec.), and operable below approximately 0.5 inch per second (12.7 mm/sec.) or above approximately 24 inches per second (609.6 mm/sec.).

Orifices are ideally sized to an area ranging between the area of a round hole of approximately 0.002 inch (0.051 mm) and approximately 0.006 inch (0.153 mm) in diameter, a optimal orifice area ranging between approximately 0.0005 inch (0.013 mm) and approximately 0.01 inch (0.254 mm) hole, an preferred orifice area ranging between approximately 0.0001 inch (0.003 mm) and approximately 0.02 inch (0.508 mm) hole, and an operable orifice area less than approximately 0.0001 inch (0.003 mm) or greater than approximately 0.02 inch (0.508 mm) in hole, though the orifice area is equivalent to the hole or diameter measurement, the orifice is not necessarily round in shape.

Optimal fluid pressure ranges between approximately 80 P.S.I. 5.6179 kg/cm²) and approximately 150 P.S.I. (10.5335 kg/cm²), preferred fluid pressure ranges between approximately 40 P.S.I. (2.809 kg/cm²) and approximately 2,000 P.S.I. (140.45 kg/cm²), and an operable fluid pressure of less than approximately 40 P.S.I. (2.809 kg/cm²) or greater than approximately 2,000 P.S.I. (140.45 kg/cm²).

Optimal fluid volume ranges between approximately 1 cc per second and approximately 6 cc per second, preferred gas volume ranges between approximately 0.05 cc per second and approximately 16 cc per second, and an operable gas volume of less than approximately 0.05 cc per second or greater than approximately 20 cc per second.

Optimal needle spacing from the needle center ranges between approximately 0.75 cm and approximately 1.25 cm, preferred needle spacing is approximately 0.4 cm and approximately 2 cm, and operable needle spacing is less than 0.4 cm or greater than 2 cm.

The hydraulic cylinder piston rod (27), is driven downward by hydraulic pressure in the upper chamber of the hydraulic cylinder (26). The hydraulic pressure is generated by a pump (33), driven by an electric motor (34), drawing fluid from a reservoir (35), through pipe (36). The fluid flow is controlled by a 4-way valve (37). When this valve is in the “down” position it directs the fluid under pressure from the pump into the upper chamber of the hydraulic cylinder through pipe (38). The fluid from the lower chamber of the hydraulic cylinder returns to the valve through pipe (39), then to the reservoir through pipe (40). When valve (37) is in the “up” position, it directs the fluid under pressure from the pump to the lower chamber of the hydraulic cylinder, and directs the return fluid from the upper chamber to the reservoir.

The gas chamber (28) receives and returns gas through a flexible pipe (41). The gas is stored at high pressure in a container (42). The gas pressure is reduced to 100 P.S.I. (7.022 kg/cm²) through a reducing valve (43), and is monitored by a pressure gauge (44). The gas then goes through a filter (45), to a 3-way valve (46), by which it can be directed to the gas chamber (28), or from the gas chamber (28) to vent directly into the atmosphere, or into a container for re-use. In the machine of FIG. 6, the initial pressure in container (42) is 1500 P.S.I. (105.3348 kg/cm²), and the filter (45) removes all particles larger than 1 micron.

Referring to FIG. 7, when during the downward motion of gas chamber (28), the needle guide plate (31) contacts the uppermost portion of the fish (47), being treated, the downward motion of the needle guide plate (31) stops, but the gas chamber (28) and the needle group (30) continue downward. This relative motion between the needle guide plate and the gas chamber operates a switch (48), which turns the electrically operated gas valve (46) to the “on” position. When the needles reach the full down position, 1 cm. above the floor of the fish tray (49), the switch (50), operated by the gas chamber, turns the gas valve (46) to the vent position. When the gas chamber returns to the full up position, the gas valve switch (50) returns to the “on” position, but no gas flows because the guide plate switch (48) is now in the “off” position.

When the machine of FIG. 6 is operating, downward motion of the needles at one inch per second the gas will expand to fully penetrate and treat all spaces between needles in less than 1 hour or up to 8 hours, depending on the density and other characteristics of the fish being treated.

Having disclosed the principles of the present invention, it will become apparent to those skilled in the art that there are many improvements, alterations, modifications, and variations from the apparatus just described. It is therefore to be understood that the present invention is not limited to the examples just described.

1. A process for injecting fluids comprising:
sizing an orifice to an accurate fluid flow rate;
combining said orifice with a needle for injecting fluid;
said fluid is a gas.

2. A process for injecting fluids comprising:
a pressurized fluid source;
said pressurized fluid flows through an orifice and needle;
said needle penetrates a permeable solid;
said orifice precisely controls the mass flow of said fluid through said needle during injection;
said fluid is gas.

3. A process for injecting fluids comprising:
a fluid at a fixed source pressure;
said fluid flows through an orifice and needle;
said orifice accurately controls the constant flow of said fluid through said needle at said fixed pressure;
said fluid is gas;
said needles penetrate a permeable solid;
said gas is injected for the purpose of treating said permeable solid.

4. A process for injecting fluids comprising:
fluid flows through an orifice while the orifice size is adjusted;
adjustment of the orifice size stops when the desired flow rate is achieved;
said orifice is used in conjunction with a needle to inject fluid.

5. A process according to any one of claims 1 to 4, wherein said orifice is sized by crimping or removing material from the orifice body to achieve said desired flow rate.

6. A process according to any one of claims 1 to 4, wherein said orifice is sized by increasing or decreasing the orifice size to obtain said desired flow rate.

7. A process according to any one of claims 1 to 4, comprising:
said pressure above the orifice is greater than about 2 times the pressure below the orifice.

8. A process according to any one of claims 1 to 4, further comprising:
said flow through the orifice reaches approximately sonic velocity.

9. A process according to any one of claims 1 to 4, wherein the flow through said orifice increases until the back pressure below the orifice reaches critical pressure;
said critical pressure is approximately 0.53 times the pressure above the orifice;
said fluid flow reaches sonic speed;
the flow rate of said fluid flowing through said orifice does not change, regardless of back pressure.

10. A process according to any one of claims 1 to 4, further comprising:
said fluid is a gas or a liquid.

11. A process according to any one of claims 1 to 4, further comprising:
said fluid is a gas or a liquid, or a combination gas and liquid as a vapor, or a combination of liquid and solid as a colloid.

12. A process according to any one of claims 2 to 3, wherein said solid is a food.

13. A process according to any one of claims 2 to 3, wherein said solid is meat or fish.

14. A process according to any one of claims 1 to 3, wherein said gas contains any one of carbon monoxide, carbon dioxide, or ozone.

15. A process according to any one of claims 1 to 4 further comprising:
a multiplicity of needles.

16. A process according to any one of claims 1 to 4, wherein said fluid flows at a continuous rate.

17. A process according to any one of claims 1 to 4, wherein said orifice is the internal diameter of the needle.

18. A process according to any one of claims 1 to 4, wherein said orifice is independent of the needle.

19. A process according to any one of claims 1 to 4, wherein said orifice is made using any one of LASER, EDM, drilling, punching, grinding, or other mechanical or non-mechanical means.

20. A process according to any one of claims 1 to 4, wherein the size of said orifice is approximately equivalent to the cross sectional area of a circle with a diameter within a range of 0.002 inch (0.0508 mm) to 0.006 inch (0.153 mm).

21. A process according to any one of claims 1 to 4, wherein the size of said orifice is approximately equivalent to the cross sectional area of a circle with a diameter within a range of 0.0005 inch (0.013 mm) to 0.01 inch (0.254 mm).

22. A process according to any one of claims 1 to 4, wherein the size of said orifice is approximately equivalent to the cross sectional area of a circle with a diameter within a range of 0.0001 inch (0.00254 mm) to 0.02 inch (0.508 mm).

23. A process according to any one of claims 1 to 4, wherein the size of said orifice is approximately equivalent to the cross sectional area of a circle with a diameter less than 0.0001 inch (0.00254 mm) or more than 0.02 inch (0.508 mm).

24. A process according to any one of claims 1 to 4, wherein the volume of said fluid through said needle ranges from 1.0 cc per second to 6.0 cc per second.

25. A process according to any one of claims 1 to 4, wherein the flow rate of said fluid through said needle ranges from 0.05 cc per second to 16 cc per second.

26. A process according to any one of claims 1 to 4, wherein the flow rate of said fluid through said needle ranges from less than 0.05 cc per second to greater than 16 cc per second.

27. A process according to any one of claims 1 to 4, wherein fluid pressure is within a range from 50 P.S.I. (3.511 kg/cm²) to 750 P.S.I. (52.6674 kg/cm²).

28. A process according to any one of claims 1 to 4, wherein said needle penetration is at a continuous rate during said injection of said fluids;
said continuous rate is between approximately 0.5 inch per second (12.7 mm/sec.) and approximately 24 inches per second (609.6 mm/sec.).

29. An apparatus for injecting fluids comprising:
fluid is injected through an orifice and needle;
wherein the size of said orifice is approximately equivalent to the cross sectional area of a circle with a diameter less than 0.01 inch (0.254 mm).

30. A product for injecting fluids comprising:
an orifice and needle;
wherein the size of said orifice is approximately equivalent to the cross sectional area of a circle with a diameter less than 0.01 inch (0.254 mm).

31. A process according to any one of claims 1 to 4, further comprising:
a multiplicity of said needles in a gas manifold with a gas delivery system and hydraulic drive system for injection into said permeable solid.

32. A process according to any one of claims 1 to 4, further comprising:
a means for preventing or removing blockages of needles.
